(19) United States
(12) Patent Application Publication
(10) Pub. No.: US 2026/0123936 A1
(43) Pub. Date: May 7, 2026

(54) TOURNIQUET WITH IMPROVED STRAP ROUTING

(71) Applicant: Composite Resources, Inc., Rock Hill, SC (US)

(72) Inventors: Michael James Casella, Fort Mill, SC (US); Kangil Cheon, San Francisco, CA (US); Jesse Arnold Fourt, Menlo Park, CA (US); Christopher Steven Hill, San Francisco, CA (US); Daniel Chien-Cheng Huang, Victorville, CA (US); Rodney Hal Monson, Waukegan, IL (US); Thomas Kai Wucherpfennig, San Francisco, CA (US)

(21) Appl. No.: 19/380,827

(22) Filed: Nov. 5, 2025

Related U.S. Application Data
(60) Provisional application No. 63/716,614, filed on Nov. 5, 2024.
(60) Provisional application No. 63/893,317, filed on Oct. 3, 2025.
(60) Provisional application No. 63/893,321, filed on Oct. 3, 2025.
(60) Provisional application No. 63/893,324, filed on Oct. 3, 2025.

(51) Int. Cl. A61B 17/132

(52) U.S. Cl. CPC A61B 17/1327

(57) ABSTRACT

A tourniquet includes a tourniquet band, a windlass, a windlass base and buckle. The tourniquet band may comprise an outer sleeve and an inner tightening strap. The windlass is operable to apply tension to the inner tightening strap to build occlusion pressure. The windlass base includes a strap guide with first and second guide slots. The strap guide is configured to allow smooth movement of the inner tightening strap during operation of the windlass to allow the windlass to smoothly build occlusion pressure beginning in the first rotation of the windlass.

### RELATED APPLICATIONS

This application claims the benefit of priority under 35 U.S.C. § 119(e) to U.S. Provisional Patent Application 63/716,614, entitled “Tourniquet with Improved Strap Routing,” filed Nov. 5, 2024, and U.S. Provisional Patent Application 63/893,317, entitled “Tourniquet with Improved Strap Routing,” filed Oct. 3, 2025, U.S. Provisional Application 63/893,321, entitled “Tourniquet with Overmolded Buckle and Improved Strap Routing,” filed Oct. 3, 2025, and United States Provisional Application 63/893,324, entitled “Tourniquet with Optimized Inner Strap and Windlass,” filed Oct. 3, 2025, which are hereby incorporated by reference herein.

### TECHNICAL FIELD

The present disclosure relates to tourniquets. Even more particularly, embodiments of the present disclosure relate to tourniquets with novel strap routing.

### BACKGROUND

Uncontrolled blood loss is a leading cause of mortality in emergency scenarios, particularly when the injured individual is alone and immediate medical assistance is unavailable. Tourniquets are widely recognized as effective devices for stemming hemorrhage from extremity injuries in such situations. For solo emergency use, the victim must be able to apply the tourniquet to his or her own arm or leg and occlude blood flow, preferably using only one hand to occlude blood flow.

Prehospital tourniquets are designed to be easily used by first responders or the injured individuals themselves to control severe bleeding before transport to a hospital. Some examples of prehospital tourniquets are described in U.S. Pat. Nos. 10,016,203, 8,888,807, 7,842,067. One notable example is the COMBAT APPLICATION TOURNIQUET (CAT) GEN 7, by Composite Resources Inc., of Rock Hill, South Carolina, USA (all tradenames, trademarks, and the like used herein are the property of their respective owners). This device is widely used by both military personnel and civilian emergency responders.

In many emergency scenarios, including those occurring during recreational activities such as hiking, rock climbing, and camping, the practicality of carrying a tourniquet is influenced by its size and weight. If a tourniquet is excessively bulky or heavy, individuals are less likely to include it in their gear, thereby reducing its availability when needed. Accordingly, there is a recognized need for a compact and lightweight tourniquet that can be conveniently packed, transported, and effectively deployed in remote or austere environments. Additionally, there is a need for tourniquet designs that are compatible with standard equipment holders commonly used by law enforcement and military personnel, such as firearm magazine pouches.

### SUMMARY

Embodiments of the present invention relate to a tourniquet system configured to restrict blood flow in a limb or other body part. According to one embodiment, a tourniquet includes a tourniquet having a tourniquet band, a windlass, a windlass base and buckle. The buckle can define an adjustment slot adapted for receiving the tail end of the tourniquet band to enable the tourniquet band to be formed into a loop.

The tourniquet band may comprise an outer sleeve and an inner tightening strap. The windlass is operable to apply tension to the inner tightening strap to build occlusion pressure. The windlass may be configured to begin building occlusion pressure upon the first rotation and may take up strap length in a single longitudinal direction or multiple longitudinal directions in various embodiments.

The windlass base includes a strap guide with first and second guide slots. The guide slots of the strap guide may be longitudinally spaced to be wider than the diameter of the windlass where the inner tightening strap passes through the windlass. The guide slots may include radiused entrance and exit features to accommodate changes in strap angle due to longitudinal displacement of the windlass. In some embodiments, at least one of the radiused entrance or exit features extends an entire depth the first guide slot or the second guide slot.

The strap guide is configured to route the inner tightening strap to and from the windlass to enable effective tightening without obstructing strap movement. In some embodiments, the inner tightening strap is routed to and from the windlass in a single-pass configuration. In other embodiments, the inner tightening strap is double routed through at least one of the windlass or the guide slots of the strap guide.

In one embodiment, the inner tightening strap is routed from the outer sleeve and through the windlass base to the windlass via the first guide slot, through the windlass, from the windlass to the second guide slot, and through the windlass base via the second guide slot. The strap guide may be configured to allow smooth movement of the inner tightening strap during operation of the windlass.

The inner tightening strap may be routed from the second guide slot back into the outer sleeve to double back on itself. In one embodiment, the inner tightening strap comprises a first end and a second end. The second end of the inner tightening strap may be anchored to the outer sleeve and the first end of the inner tightening strap may be anchored back to the inner tightening strap. In some embodiments, the second end of the inner tightening strap is anchored back to the inner tightening strap away from the second end of the inner tightening strap.

The outer sleeve may comprise a first end and a second end. The second end of the inner tightening strap may be anchored to the second end of the outer sleeve. The windlass base may be coupled to the first end of the outer sleeve. In some embodiments, the windlass base is overmolded to the outer sleeve.

According to one embodiment, the strap guide comprises a first longitudinal portion, a second longitudinal portion; a first lateral portion spanning between the first longitudinal portion and the second longitudinal portion, a second lateral portion spanning between the first longitudinal portion and the second longitudinal portion and an intermediate lateral portion spanning between the first longitudinal portion and the second longitudinal portion, the intermediate lateral portion located between the first lateral portion and the second lateral portion. The first longitudinal portion, the second longitudinal portion, the first lateral portion and the intermediate lateral portion may define the first guide slot. The first longitudinal portion, the second longitudinal portion, the second lateral portion and the intermediate lateral portion define the second guide slot.

According to one embodiment, the strap guide may further comprise longitudinal and lateral portions forming defined slot geometries, including tilted lateral guide walls relative to a z-axis perpendicular to the tourniquet's longitudinal and lateral axes.

The tourniquet may further include a securing mechanism to prevent unintentional unwinding of the windlass. In some embodiments, the securing mechanism comprises one or more hooked catches, and may include a rotatable clip—such as a metal wire form—configured to engage the catches and lock the windlass in place. The buckle and securing mechanism may be integrated with the windlass base or overmolded or otherwise coupled to the tourniquet band.

Embodiments can enable a compact, efficient, and user-friendly tourniquet design suitable for emergency medical use, including self-application and deployment in austere environments.

### BRIEF DESCRIPTION OF THE DRAWINGS

The drawings accompanying and forming part of this specification are included to depict certain aspects of the invention. A clearer impression of the invention, and of the components and operation of systems provided with the invention, will become more readily apparent by referring to the exemplary, and therefore non-limiting, embodiments illustrated in the drawings, wherein identical reference numerals designate the same components. Note that the features illustrated in the drawings are not necessarily drawn to scale.

FIG. 1 is a diagrammatic representation of one embodiment of a tourniquet in a stretched-out/unfurled configuration.

FIG. 2 is a diagrammatic representation of one embodiment of a tourniquet with the band coiled and arranged in a compact and organized manner.

FIG. 3 is a diagrammatic representation of one embodiment of a windlass base.

FIG. 4 is a diagrammatic representation of one embodiment of a first end of tourniquet.

FIG. 5 is a diagrammatic representation providing a side view of one embodiment of the first end of a tourniquet.

FIG. 6 is a diagrammatic representation providing a cross-sectional representation of the embodiment of FIG. 5.

FIG. 7 is a diagrammatic representation of a second end of tourniquet.

FIG. 8 is a diagrammatic representation providing a cutaway representation of one embodiment of a first end of tourniquet 10.

FIG. 9 is a diagrammatic representation providing a cutaway representation of one embodiment of the first end of a tourniquet with the outer sleeve hidden.

FIG. 10 is a diagrammatic representation of another embodiment of routing an inner strap.

FIG. 11 is a diagrammatic representation of yet another embodiment of routing an inner strap.

FIG. 12A and FIG. 12B illustrate yet another embodiment of routing an inner strap.

### DETAILED DESCRIPTION

The disclosure and various features and advantageous details thereof are explained more fully with reference to the exemplary, and therefore non-limiting, embodiments illustrated in the accompanying drawings and detailed in the following description. It should be understood, however, that the detailed description and specific examples, while indicating the preferred embodiments, are given by way of illustration only and not by way of limitation. Descriptions of known techniques may be omitted so as not to unnecessarily obscure the disclosure in detail. Various substitutions, modifications, additions and/or rearrangements within the spirit and/or scope of the underlying inventive concept will become apparent to those skilled in the art from this disclosure.

Embodiments of the present disclosure relate to tourniquet systems configured to restrict blood flow to an extremity, such as an arm or leg, in emergency medical situations. Certain embodiments incorporate a strap guide mechanism that facilitates enhanced routing of the inner tensioning strap. This improved routing architecture supports a more compact and efficient tourniquet design, enabling reductions in overall device size without compromising functional performance.

Before proceeding, some additional context may be helpful. As discussed, there is a desire for smaller, more conveniently carried tourniquets that fit in existing equipment holders, or in new storage or transportation locations not previously feasible with existing tourniquet sizes. However, efforts to reduce the physical dimensions of conventional tourniquet designs encounter several functional limitations. First, it is preferable that the constriction band maintain a minimum width to achieve effective vascular occlusion; industry standards generally recognize 1.5 inches as the minimum acceptable width to achieve effective occlusion pressure (e.g., the CAT GEN 7 tourniquet utilizes a 1.5-inch-wide band). Additionally, the band is preferably long enough to accommodate the limb circumference of a broad user population. For instance, the 99th percentile leg circumference is approximately 30.7 inches, necessitating a band length that exceeds this measurement to ensure universal applicability (e.g., the CAT GEN 7 features a 37.5-inch-long band). Consequently, while there is a clear desire to reduce the overall package size of tourniquets, it remains preferable that the band dimensions—both in width and length—remain consistent with established designs to preserve efficacy.

Additionally, the hardware of prior designs cannot simply be miniaturized to achieve a smaller package size as doing so would result in high torques. Prehospital tourniquets typically utilize a dual-component band structure comprising an outer sleeve and an inner tensioning strap. The inner strap is anchored within and slidable relative to the outer sleeve. The inner strap emerges from the outer sleeve and is routed through a windlass mechanism to facilitate incremental tightening. In many designs, including the CAT GEN 7, the inner strap forms a continuous loop within the outer sleeve and is double-routed through various hardware slots—passing through the slots twice. Specifically, the strap extends from the free end of the outer sleeve to the buckle, loops through a slot in the buckle, and returns to the free end, where both ends are secured. This configuration forms a loop and results in two overlapping inner strap layers. Both layers of the inner strap emerge from the outer sleeve to pass through the windlass, effectively doubling the strap's passage through the windlass slot.

The windlass is typically positioned above a base plate that rests against the victim when the tourniquet is applied. The base plate includes narrow strap routing slots—similar to the slots of a tri-glide slide—designed to secure the base plate to the strap and retain the position of the base plate on the strap. In the CAT GEN 7, for example, the base plate incorporates dual-slot arrangements at both ends, resembling tri-glide slides. At the first pair of slots, the tourniquet band (outer sleeve and inner band) passes from the front of the base plate to the rear of the base plate through one slot and reemerges through the next slot to weave tightly behind a first plastic bar between the first pair slots. At the second pair of slots, the tourniquet band (outer sleeve and inner band) passes from the front of the base plate to the rear of the base plate through one slot and reemerges through the next slot to weave tightly behind a plastic bar between the second pair of slots. Because the inner strap is double-routed, the inner strap passes through each of these tri-glide slide-type arrangements twice (e.g., once running from the tip of the band to the buckle and once on the return from the buckle to the tip of the tourniquet band).

Weaving the tourniquet band through the strap routing slots in the base plate results in substantial friction, which impedes the sliding motion of the inner strap relative to the outer sleeve during windlass rotation. This friction can require high windlass torque to overcome. Further, with some tourniquets, the user must turn a windlass several turns before the tourniquet begins building pressure around the body part. Some tourniquets also have a pressure function where, once pressure starts to build, it builds quickly for the first several turns, then flattens out, and then builds sharply, making it difficult for the user to keep turning the windlass.

Simply miniaturizing the hardware, such as reducing the size of the base plate and the windlass would exacerbate this friction, thereby requiring significantly higher torque to operate the windlass. For example, shrinking the hardware of a tourniquet, such as the CAT GEN 7, would decrease the size of the base plate slots, further increasing friction, thereby requiring high windlass torque to overcome. Moreover, shortening the windlass rod to reduce overall device size diminishes mechanical leverage, further complicating torque generation. These factors would collectively degrade the tourniquet's ability to achieve effective occlusion pressure across a wide range of limb sizes.

Further, certain tourniquet designs require multiple windlass rotations before any meaningful pressure is exerted on the limb. In some cases, pressure builds quickly for the first several turns, then flattens out, and then builds sharply. This behavior can make it difficult for the user to keep turning the windlass.

Embodiments of the present application provide tourniquets with improved inner strap routing and complementary features that enable a reduced overall size without compromising performance relative to prior prehospital tourniquets. In some embodiments, the inner strap is single-routed (single pass) such that the inner strap is routed through the slots of one or more pieces of hardware once. For example, the inner strap may be routed such that it passes through a windlass only once.

Single-pass routing of the inner strap can allow the tourniquet to be smaller by reducing the doubling up of the inner strap. The improved strap routing also allows hardware components to be smaller. Further, the improved inner strap routing allows the bulk of the tourniquet to be concentrated at one end.

Further, embodiments may maintain a more consistent pressure profile while requiring lower baseline torque and minimizing sudden torque spikes. Such characteristics enhance usability, particularly for individuals with limited strength—such as those self-administering the device—or for users with minimal training, thereby improving the overall accessibility and effectiveness of the tourniquet in emergency scenarios.

The improved inner strap routing can reduce or eliminate the need for the working segment of the inner strap to be tightly woven through strap routing slots in the base plate, thereby reducing hardware-induced friction. This reduction in hardware-induced friction facilitates smoother strap movement during windlass activation, thereby lowering the torque required to achieve effective tightening. Furthermore, by reducing or eliminating pinching of the inner strap at the strap routing slots, embodiments promote more uniform strap motion, which in turn mitigates sudden pressure spikes and torque surges.

The improved inner strap routing of some embodiments improves the pressure build function of the tourniquet (e.g., lower torque and finer tuning). In particular, the inner strap is routed such that the windlass begins to build pressure within the first turn and continuously builds pressure as it is turned. The more gradual pressure build allows a user to more finely tune the final applied pressure. With lower required input torque, a user is more likely to achieve occlusion pressure. With finer tuning, the user is less likely to overtighten the tourniquet with extra tension beyond what is required for occlusion.

Tourniquets according to the present disclosure can be approximately 30% smaller than the CAT GEN 7 for the same width and length of band, while achieving similar or improved performance. This allows, for example, the tourniquet to fit in some sizes of standard equipment holders worn by law enforcement and military personnel (e.g., in firearm magazine holders).

Embodiments according to the present disclosure may incorporate a band buckle and an inner strap latch as a single part.

Embodiments may include a robust strap retention clip formed of metal or similar strong material. Such a retention clip may be easier to manipulate and secure compared with a hook and loop strap, while also being less likely to catch on or scratch outside materials such as the pockets of law enforcement uniforms.

Embodiments according to the present disclosure may also include features that enhance manufacturability. For example, embodiments may include an integrated buckle and latch over-molded to the outer band, or a non-over molded version that utilizes molded features to aid assembly using heat or ultrasonic welding.

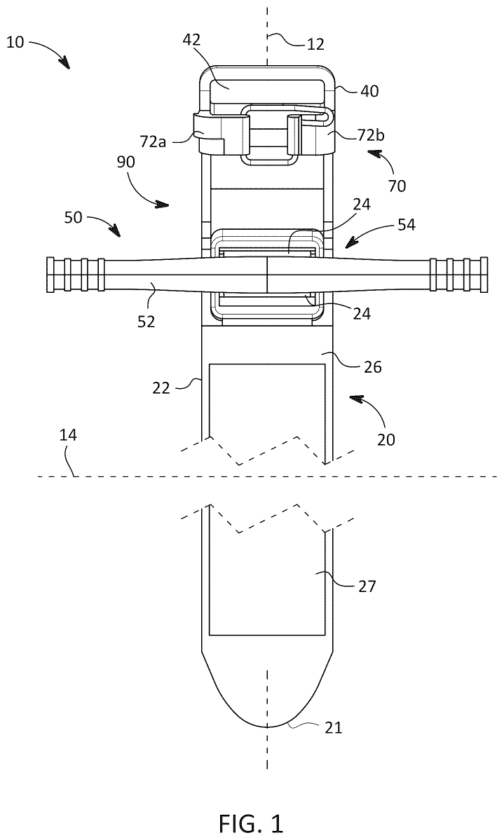

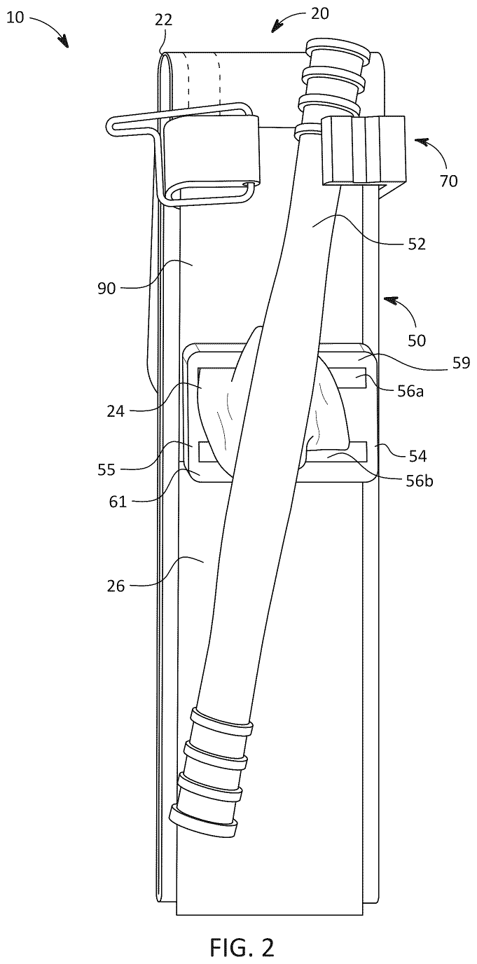

FIG. 1 is a diagrammatic representation of one embodiment of a tourniquet 10 in a stretched-out configuration. For reference, FIG. 1 illustrates the longitudinal axis 12 and lateral axis 14. The z-axis refers to the axis that is perpendicular to the longitudinal axis 12 and lateral axis 14 of the tourniquet. FIG. 2 is a diagrammatic representation of one embodiment of tourniquet 10 in a folded configuration suitable for storage or transport.

With reference to FIGS. 1-2, tourniquet 10 comprises a tourniquet band 20, a buckle 40, a strap guide, a tightening mechanism 50 and a securing mechanism 70. In the illustrated embodiment, buckle 40, strap guide 54, and securing mechanism 70 are integrated as portions of an integrated base plate 90 to which one end of band 20 is anchored. In one embodiment, windlass 52 and base plate 90 are formed of any suitable materials, such as a hard plastic (for example, but not limited to, a Nylon injected molded plastic), metal, composite material or other materials. In other embodiments, one or more of buckle 40 or securing mechanism 70 is provided as a separate part from the base plate 90 that includes strap guide 54. By way of example, but not limitation, a base plate may include a strap guide 54 and securing mechanism 70, while the buckle 40 is provided as a separate part.

Tourniquet band 20 includes an outer sleeve 22 defining an interior area (e.g., a pocket or other inner area) in which an inner tightening strap 24 is disposed. Outer sleeve 22 comprises a longitudinally extensive material having a first end and a second end (a tail end 21). Buckle 40 is coupled to the first end of outer sleeve 22 and includes an adjustment slot 42 through which tail end 21 of band 20 can be passed, enabling the formation of a loop for encircling a body part. This loop can be pulled tight around the body part.

Band 20 includes fastening mechanisms so that band 20 can be secured to itself after being looped through buckle 40. In one embodiment, the outer surface 26 of outer sleeve 22 includes an area 27 of both hook and loop structures. More preferably, outer surface 26 comprises both hook structures and loop structures along substantially the entire length of outer sleeve 22 between the second end 21 of outer sleeve 22 and an opening where inner strap 24 is exposed. Thus, when the second end of outer sleeve 22 is looped through buckle 40, the outer surface 26 may be applied to itself, thereby securing the position of outer sleeve 22. By way of example and not limitation, the outer surface of outer sleeve 22 comprises a length of OMNI-TAPE® (Velcro Industries B.V., Amsterdam, Netherlands), wherein the fastening surface comprises both hook and loop structures on the outer surface 26. The use of a combination of both hook and loop structures on the outer surface 26 of outer sleeve 22 provides the advantage of the tourniquet being quickly adjustable when in use to accommodate a variety of size appendages, as for example, from a person's thigh to a person's forearm. In addition, or in the alternative, to using hook and loop material as a fastener, outer sleeve 22 may be fitted with other types of fasteners, such as, but not limited to rigid hooks and loops, buttons, snaps, transverse straps, or other fastening mechanisms.

Tightening mechanism 50 is operable to apply tension to inner tightening strap 24. According to one embodiment, tightening mechanism 50 comprises a windlass 52 that can be rotated to tension inner tightening strap 24. Strap guide 54 facilitates routing of inner tightening strap 24 between the windlass and the internal compartment of outer sleeve 22.

Securing mechanism 70 includes features to prevent unintentional unwinding of windlass 52, thereby maintaining the applied tension in the inner tightening strap 24 and ensuring sustained occlusion pressure.

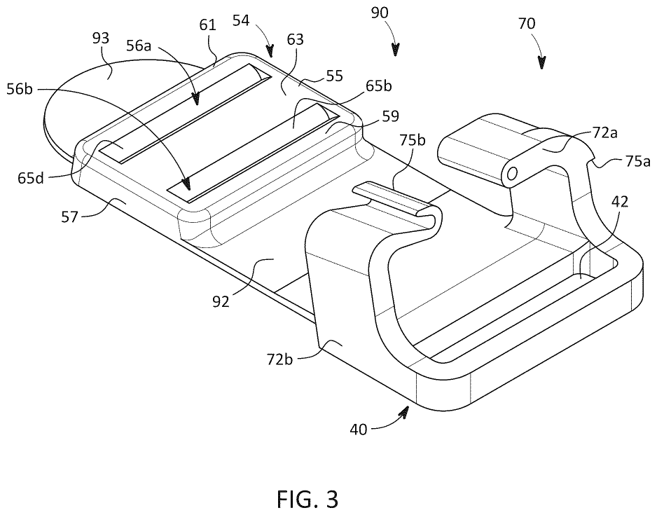

FIG. 3 is a diagrammatic representation of one embodiment of base plate 90. As discussed, base plate 90 may be anchored to a first end of tourniquet band 20. In the illustrated embodiment, base plate 90 includes a base member 92 that extends longitudinally from a first end to a second end. Buckle 40 extends longitudinally from a first end of base member 92 and forms adjustment slot 42 through which tail end 21 of tourniquet band 20 may be looped. Securing mechanism 70 comprises a pair of hooked catches 72a, 72b that extend outward from base member 92 at the first end of base member 92 adjacent to buckle 40. Strap guide 54 also extends outwardly from base member 92 and comprises longitudinal and lateral walls that define guide slots 56a, 56b for routing inner strap 24 to/from windlass 52. Base plate 90 includes an extension 93 proximate to the second end of base plate 90. Extension 93 may extend into the interior pocket of outer sleeve 22.

In one embodiment, strap guide 54 comprises a first longitudinal portion 55 and a second longitudinal portion 57 interconnected by a first lateral portion 59, a second lateral portion 61 and an intermediate lateral portion 63 to form guide slots 56a, 56b. First lateral portion 59, second lateral portion 61 and intermediate lateral portion 63 can comprise lateral strap guide walls (e.g., example strap guide walls are 65b, 65d indicated in FIG. 3, further examples of strap guide walls 65a, 65c are indicated in FIG. 6).

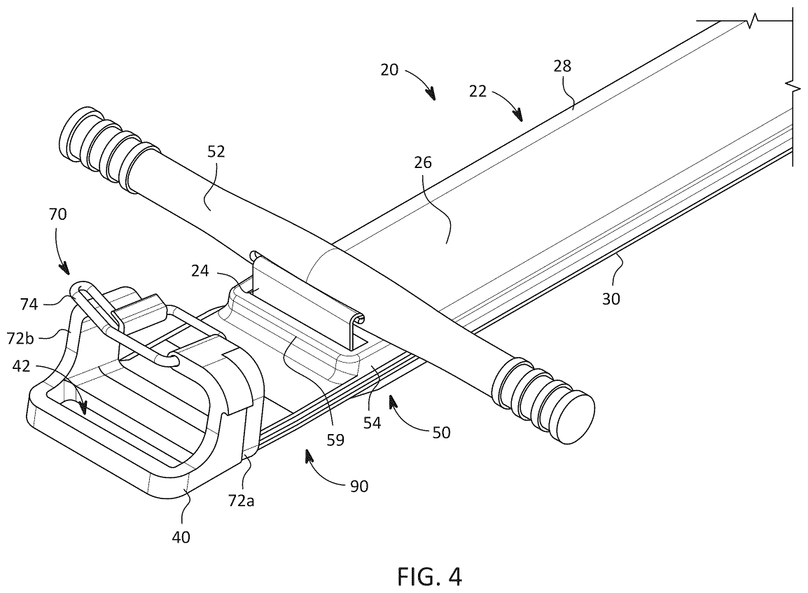

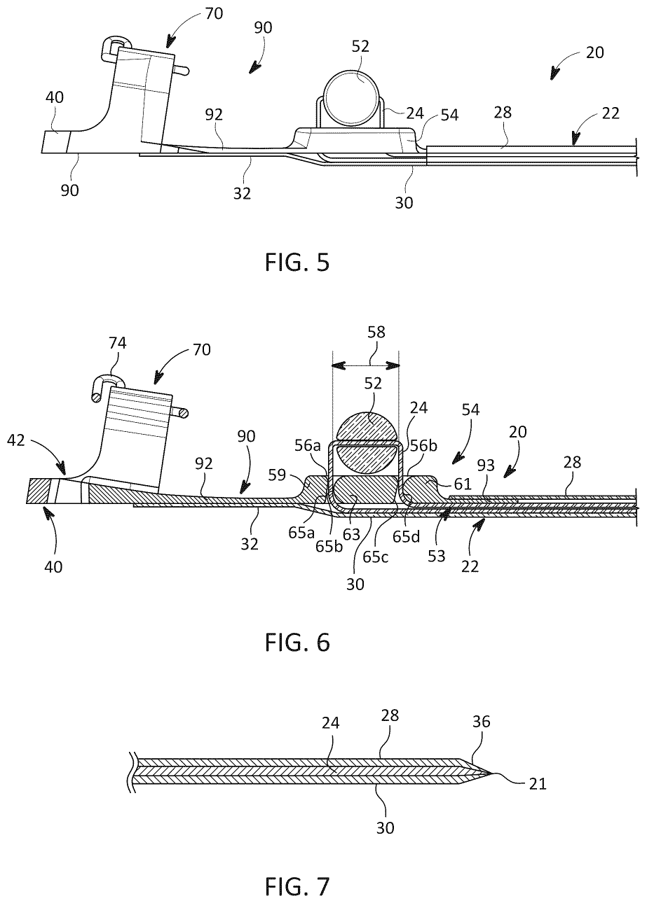

Referring now to FIGS. 4-7, FIG. 4 is a diagrammatic representation of one embodiment of a first end of tourniquet 10, FIG. 5 is a diagrammatic representation providing a side view of one embodiment of a first end of tourniquet 10, FIG. 6 is a diagrammatic representation providing a cross-sectional representation of the embodiment of FIG. 5, and FIG. 7 is a diagrammatic representation of a second end of tourniquet 10.

In accordance with one embodiment, outer sleeve 22 is formed of one or more panels of materials such as, but not limited to, flat weave nylon binding tape. In the illustrated embodiment, sleeve 22 is formed of two panels comprising an outer panel 28 and an inner panel 30. The edges of panels 28 and 30 are connected, as for example, by sewing, glueing, stapling, clamping, or heat/ultra-sound (sonic) welding, or combinations thereof to form an interior area in which inner strap 24 is disposed. In another example embodiment, outer sleeve 22 is formed of a single piece of material, such as by way of example and not limitation, a piece of material that is folded over and seamed to form the interior area.

The inner portion of outer sleeve 22 extends past the outer portion to provide an extended portion 32. For example, inner panel 30 may extend past outer panel 28 to form extension 32. In the illustrated embodiment of FIG. 6, hardware member 90 is coupled to the extension 32 at the first end of outer sleeve 22. In an even more particular embodiment, hardware member 90 is over-molded onto the extension 32 at the end of outer sleeve 22. A portion of base plate 90 (e.g., portion 93) may extend into the interior space of outer sleeve 22.

In one embodiment of overmolding, extension 32 is positioned in the mold and secured with clamping features. Material is introduced into the mold at a temperature calibrated to promote molecular bonding between the injected plastic and the tourniquet band materials. The materials selected for base member 92 and outer sleeve 22 may possess similar melt temperatures to facilitate cohesive bonding during the molding process. For instance, all components may be fabricated from or incorporate Nylon, enabling thermal fusion and enhancing the mechanical integration of the hardware with the outer sleeve.

Inner strap 24 comprises a length of material with a tensile strength and relatively low elasticity to support the forces needed to apply occlusion pressure to a body part. According to one embodiment, inner strap 24 is formed from a length of polyester twill weave material. In another embodiment, inner strap 24 comprises a length of ultra-high-molecular-weight polyethylene, such as DYNEEMA. Other materials, including but not limited to nylon, may also be used.

Inner strap 24 preferably has frictional characteristics allowing it to slide within the interior space of the outer sleeve 22 when a tensile force is applied to the inner strap 24. The interior space of the outer sleeve 22 may include a substance, such as a powder or other lubricant or a sheet of plastic or other relatively low friction material, to assist with the frictional characteristics between the surfaces of the inner strap 24 and outer sleeve 22.

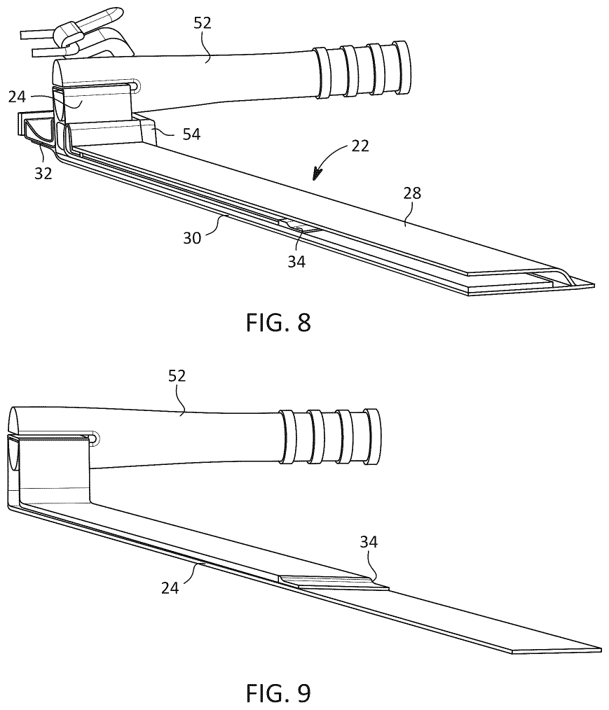

According to one embodiment, inner strap 24 includes a first end 34 (FIG. 8, FIG. 9) and a second end 36 (FIG. 7) distal from first end 34. A portion of inner strap 24 is anchored to outer sleeve 22 as for example, by sewing, glueing, stapling, clamping, or heat/ultra-sound (sonic) welding, or another anchoring mechanism. According to an even more particular embodiment, only the second end 36 of inner strap 24 is anchored to outer sleeve 22. For example, the second end 36 of inner strap 24 may be anchored to the outer sleeve 22 at the second end of outer sleeve 22 (i.e., at the tail end 21 of outer sleeve 22) or at another location. The remainder of inner strap 24, including first end 34, is not anchored to outer sleeve 22. In one embodiment, the first end 34 of inner strap 24 is anchored back to inner strap 24 between tail 21 of tourniquet band 20 and where inner strap 24 emerges from outer sleeve 22.

With reference to FIG. 6, outer sleeve 22 forms an opening 53 through which inner strap 24 emerges to pass between the extended portion of inner panel 30 and base member 92 and, in particular, behind strap guide 54. Inner strap 24 is routed from the interior space of outer sleeve 22, through slot 56a of strap guide 54, through windlass 52, through slot 56b of strap guide 54, and back into the interior space of outer sleeve 22 to double back on itself. In another embodiment, the routing through strap guide 54 may be reversed such that the double-backed portion of inner strap 24 is to the inner side (closer to the victim when in use).

First end 34 of inner strap 24 (FIG. 8, FIG. 9) is anchored back to inner strap 24 at a location along the length of inner strap 24 away from the second end of inner strap 24, as for example by sewing, glueing, stapling, clamping, or heat/ultra-sound (sonic) welding, or another anchoring mechanism. In one embodiment, first end 34 is anchored to inner strap 24 at the midway point of inner strap 24. In other embodiments, first end 34 may be anchored elsewhere along the length of inner strap 24, including at the second end. Referring still to FIG. 6, in the embodiment illustrated, inner strap 24 is routed once through each guide slot 56a, 56b of strap guide 54 and windlass 52 as a single-routed strap—that is, inner strap 24 is routed to create one strap layer through windlass 52 and the slots of strap guide 54.

Upon rotation of the windlass 52 by the user, the windlass engages and draws the inner tightening strap 24, thereby initiating the formation of a coil. Continued rotation of windlass 52 incrementally increases tension within the strap, resulting in circumferential compression of the tourniquet band and progressive occlusion of blood flow. In one embodiment, the incorporation of two closely spaced strap guide slots (slot 56a and slot 56b) combined with a taut inner strap 24 (i.e., minimal slack prior to rotation), enables the tourniquet to begin building occlusive pressure upon the first rotation of the windlass, thereby improving responsiveness and efficiency.

Slots 56a, 56b help guide inner strap 24 to windlass 52 in a manner that promotes low-friction movement during operation. For example, slots 56a, 56b can be designed not to pinch inner strap 24 and may include radiused corners to promote smooth flow of inner strap 24. This guided routing can help ensure that the strap transitions smoothly from the interior of the outer sleeve to the windlass, allowing the coil to form immediately upon initial rotation and inner strap 24 to flow freely as windlass 52 is turned. Thus, according to some embodiments, windlass 52 immediately begins to build a coil that builds pressure when it is turned without requiring several initial turns and continuously builds the coil to produce a gradual pressure build.

As will be appreciated, if slots 56a and 56b are too large, inner strap 24 may bind as the coil is wound or released. Thus, the size (length, width, depth) of slots 56a and 56b can be selected to prevent binding, prevent the coil from propagating down band 20, and to promote easy resetting for the device. Further, to prevent pinching of strap 24, the minimum length of the slots (i.e., parallel to the longitudinal axis 12 of FIG. 1) is at least equal to the thickness of inner strap 24 that passes through the slot when the inner strap has not been tensioned by the windlass.

Strap guide slots 56a and 56b may be further geometrically configured to accommodate variations in the exit angle of the inner tightening strap 24, which may result from longitudinal displacement of the windlass 52 relative to strap guide 54. The slots may incorporate features to facilitate smooth strap movement under dynamitic conditions, including but not limited to, radiused features at their entry points or exit points. The radiused features of a slot may extend part of the depth of a slot or the entire depth of the slot. The radiuses may be selected to prevent or minimize shearing of the inner strap 24.

In the illustrated embodiment, the lateral face 65a of lateral portion 59 includes a predominantly straight segment extending along the majority of the depth of slot 56a, with radiused transitions at both the inner (closer to the victim when the tourniquet is applied) and outer (further from the victim when the tourniquet is applied) ends. This straight segment may be angularly offset from the z-axis—such as being inclined toward the windlass 52 from the inner end of slot 56a (i.e., the side positioned closer to the user's body when the tourniquet is applied) to the outer end (i.e., the side farther from the body). Further, in the illustrated embodiment, the lateral face 65b of intermediate lateral portion 63 may be continuously radiused along the full depth of slot 56a. The lateral face 65c of intermediate lateral portion 63 may also include a straight segment along most of the depth of slot 56b, with radiused ends, and may be angled to correspond with the inclination of lateral face 65a relative to the z-axis. In one embodiment, the straight segments of lateral faces 65a and 65c are tilted between approximately 5 to 15 degrees from the z-axis, with a preferred tilt angle of approximately 10 degrees (e.g., within a range of 9 to 11 degrees). Additionally, the lateral face 65d of second lateral portion 61 may be radiused along the entire depth of slot 56b to further promote low-friction strap movement. In some embodiments, the radiuses of lateral faces 65b and 65d match.

In addition, or in the alternative, slots 56a and 56b are spaced to create a span 58 in inner strap 24 as it exits/enters strap guide 54 that is slightly wider than the diameter of windlass 52 where inner strap 24 passes through windlass 52. By way of example, but no limitation, span 58 may be 5%-10% larger than the diameter of windlass 52 where inner strap 24 passes through windlass 52. In one embodiment, the radiuses on the outer sides of the slots are selected to maintain this span as windlass 52 is displaced longitudinally, at least through a selected range of longitudinal displacement.

Embodiments of the inner strap routing of the present disclosure can promote a more compact size compared to prior tourniquets. As discussed, inner strap 24 may be anchored back to itself away from the second end of inner strap 24. There may thus be less doubling back of the inner strap compared to the GEN CAT 7. As such, tourniquet band 20 may be thinner than the tourniquet band of the CAT GEN 7 along a portion of the tourniquet band 20, even if inner strap 24 is the same thickness as or even slightly thicker than the inner strap of the CAT GEN 7. Moreover, the bulky areas of the tourniquet, such as hardware and where the inner strap is doubled back, are concentrated in one area. This can allow a tourniquet band of the same width and length as that of the CAT GEN 7 to be folded into a smaller package size compared to the CAT GEN 7.

Further, as discussed, inner strap 24 may double back on itself at strap guide 54. The routing creates a pulley effect where windlass 52 and inner strap 24 take up length in one longitudinal direction relative to the windlass, instead of two. Moreover, the strap routing can reduce or eliminate areas where the strap is pinched or tightly woven through hardware, thereby reducing friction and allowing windlass 52 to build pressure with less torque. As such, a smaller windlass 52 and securing mechanism 70 can be used (compared to the CAT GEN 7).

With reference to FIGS. 3 and 4, securing mechanism 70 preferably comprises a pair of opposing hooked catches 72a and 72b that extend outward from a base member 92 and are set substantially transverse to the longitudinal axis of tourniquet 10. More particularly, hooked catches 72a, 72b are preferably sized to cup or hold the windlass 52, or a portion thereof, and prevent it from unwinding. Accordingly, hooked catches 72a, 72b are sufficiently stiff to provide adequate resistance against the tensile force within inner strap 24, as transferred to the hooked catches 72a, 72b by the windlass 52. With two hooked catches, the user can rotate windlass 52 in either direction to apply pressure. When the user has rotated the windlass to apply sufficient pressure to the body part, the user can hook the end of the windlass 52 under the hooked catch 72a, 72b that will prevent windlass 52 from unwinding.

While two hooked catches 72a, 72b are illustrated, other embodiments may use a single hooked catch. For a single hooked catch, the user rotates the windlass in the proper direction to allow the tension in the inner strap 24 to be resisted by the single hooked catch once winding of the windlass and tensioning of the inner strap 24 is completed.

Embodiments may include structures to further secure windlass 52. These structures can allow the user to secure the windlass 52 and move about (or be moved by another person) with less concern of the windlass 52 dislodging from the hooked catches 72a, 72b and unwinding. In one embodiment, tourniquet 10 comprises a rigid clip 74, such as is formed of metal or plastic. In an even more particular embodiment, clip 74 is a metal wire form clip.

Clip 74 is coupled to hooked catch 72a and is configured to rotate into engagement with hooked catch 72b. Hooked catch 72a may incorporate a retaining feature 75a (FIG. 3), such as a longitudinal ridge or other detent or feature, to hold clip 74 in its fully open position and prevent premature closure of clip 74. Upon application of a sufficient force, the resilience of clip 74 or hooked catch 72a allows clip 74 to release from the retaining feature and rotate to a closed position. This hooking may include clip 74 sliding into a notch, groove or other indent or being captured by a retaining feature 75b (FIG. 3), such as a tooth or other detent to lock clip 74 in position. In some embodiments, hooked catch 72b includes a lead-in or other feature to help guide hook clip 74 into the correct position.

Other mechanisms may be used to help secure windlass 52, such as, but not limited to, a transversely oriented strap having hook and loop fastening portions or an elastic band that hooks over hooked catch 72a and hooked catch 72b. Helpfully, a user may rest the windlass in one of the hooked catches without deploying the structure that further secures the windlass, conveniently allowing the user to further tighten the tourniquet by unhooking the windlass, rotating the windlass, and re-hooking the windlass. Furthermore, the structures that further secure the windlass may be locked or unlocked one-handed, increasing the circumstances in which a user can lock the structure or increase tension on an already-locked tourniquet, for either a self-application or application to a different victim.

In operation, a user applies the tourniquet to a limb. In one embodiment, the tourniquet is stored in a folded-up configuration with the tail end 21 of band 20 already through buckle 40 and secured to the outer surface of the band to form a loop. In another embodiment, if the tail end of band 20 is free, the user loops the free end of band 20 through slot 42 of buckle 40 to create a loop and secures the tail end to the outer surface of band 20 using the hook and loop material. In either case, the loop may be formed prior to the tourniquet being applied to the limb. The user can then slide the preformed loop over the limb, unsecure the tail end of band 20, pull tail end of band 20 to achieve an initial cinch, and resecure band 20 to itself. In other cases, it may not be possible or optimal to use a preformed loop. In such a case, the user may undo the preformed loop if present, thread the free end of band 20 of the tourniquet around the limb, route the free end of band 20 through slot 42 of buckle 40 to create a loop around a body part, pull the free end to tighten the loop around the body part and secures band 20 to itself.

Once the initial cinch is achieved and band 20 secured to itself, the user winds windlass 52 to tighten inner strap 24, thereby causing the tourniquet to apply circumferential pressure to the body part. When a sufficient pressure is achieved, the user hooks the end of the windlass 52 under hooked catch 72a or hooked catch 72b and locks clip 74.

As discussed above, a strap guide, such as strap guide 54, can be used to guide an inner strap to the tourniquet windlass in a manner that promotes low-friction movement during operation. Such strap guides may be used with a variety of inner strap routing schemes.

Some detail is omitted in some of the figures for simplicity. However, it will be appreciated that each embodiment may comprise an outer sleeve, windlass hooks and other features discussed above.

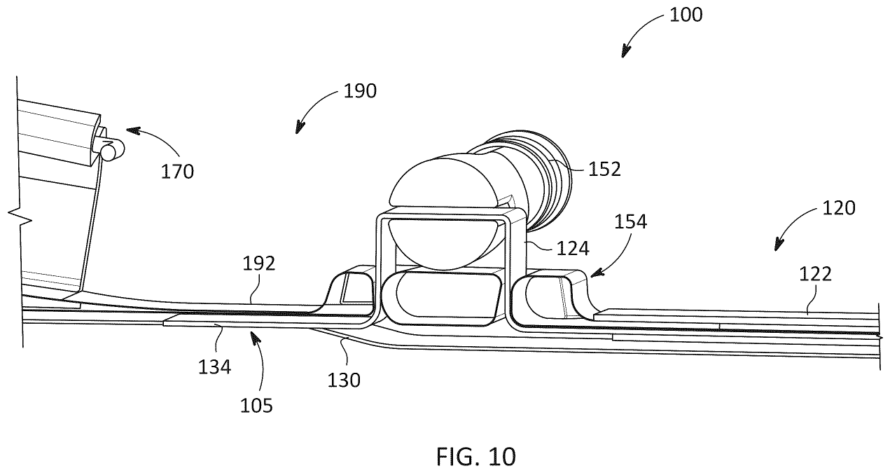

In the embodiment FIG. 10, a tourniquet 100 comprises a tourniquet band 120 and hardware, such as a tightening mechanism (e.g., windlass 152), a strap guide 154, a securing mechanism 170, and buckle (not shown). Two or more of the securing mechanism 170, strap guide 154 and or buckle may be integrated into a base plate 190, which may be similar to base plate 90.

Tourniquet band 120 may comprise an outer sleeve 122 and an inner strap 124. Outer sleeve 122 may be constructed as discussed above with respect to outer sleeve 22. The inner band 124 includes a first end 134 and a second end (not shown). A portion of inner strap 124 is anchored to outer sleeve 122 as for example, by sewing, glueing, stapling, clamping, or heat/ultra-sound (sonic) welding, or another anchoring mechanism. According to an even more particular embodiment, the second end of inner strap 124 is anchored to outer sleeve 122 at, for example, the second end of outer sleeve 122 (i.e., at the tail end of outer sleeve 122).

In one embodiment, inner strap 124 exits the outer sleeve 122 of tourniquet band 120, passes through the strap guide 154, which may be similar to strap guide 54, through the windlass 152, through the next slot in the strap guide 154 and into an area 105 between the lower cover portion 130 of outer sleeve and base member 192. End 134 of inner strap 124 may be welded using heat or ultrasonic welding or otherwise anchored to cover portion 130 or the inner side of base member 192.

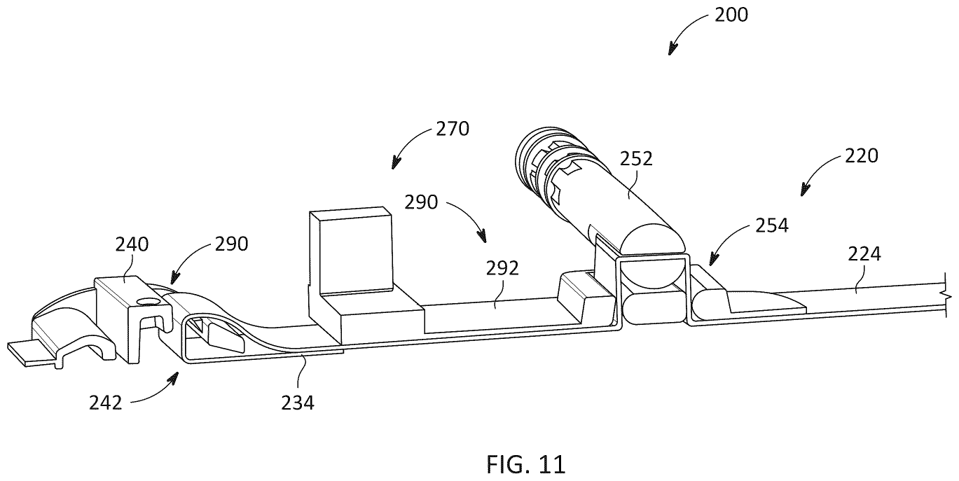

In the embodiment FIG. 11, a tourniquet 200 comprises a tourniquet band 220 and hardware, such as a tightening mechanism (e.g., windlass 252), a strap guide 254, a securing mechanism 270, and buckle 240. In the illustrated embodiment, strap guide 254 and securing mechanism 270, such as catch hooks with a clip or other securing mechanism, may be integrated into a base plate 290, which may be similar to base plate 90 without buckle 40. Buckle 240 is provided as a separate piece of hardware from the windlass base.

The tourniquet band 220 may comprise an outer sleeve (not shown) and an inner strap 224. The outer sleeve may be constructed as discussed above with respect to outer sleeve 22. The inner band 224 includes a first end 234 and a second end (not shown). A portion of inner strap 224 is anchored to the outer sleeve as for example, by sewing, glueing, stapling, clamping, or heat/ultra-sound (sonic) welding, or another anchoring mechanism. According to an even more particular embodiment, the second end of inner strap 224 is anchored to the outer sleeve at, for example, the second end of outer sleeve (i.e., at the tail end of outer sleeve).

In one embodiment, inner strap 224 exits the outer sleeve of tourniquet band 220, passes through the strap guide 254, which may be similar to strap guide 54, through the windlass 252, through the next slot in the strap guide 254 and into an area between the lower cover portion of outer sleeve and base member 292. The lower cover portion of outer sleeve and inner strap 224 loop through and attachment slot 242 of buckle 240. The end 234 of inner strap 224 that looped through buckle 240 may be anchored back to inner strap 224 at a location along the length of inner strap 224 between windlass hardware base and buckle 240, as for example by sewing, glueing, stapling, clamping, or heat/ultra-sound (sonic) welding, or another anchoring mechanism. The portion of the outer sleeve that looped through attachment slot 242 may double back and be attached back to the outer sleeve between buckle 240 and the windlass base (e.g., base plate 290).

In another embodiment, inner strap 224 may be double layered to both sides of the windlass but only passes through the windlass once. The end 234 that loops through attachment slot 242 of buckle 240 may be anchored back to inner strap 224 or the outer sleeve at a location between windlass base and the tail end of the band 220 (e.g., to the right of the windlass in FIG. 11) or at the free end the tourniquet band.

In any event, in the embodiment of FIG. 11, turning the windlass tightens inner strap 124 to both sides of the windlass.

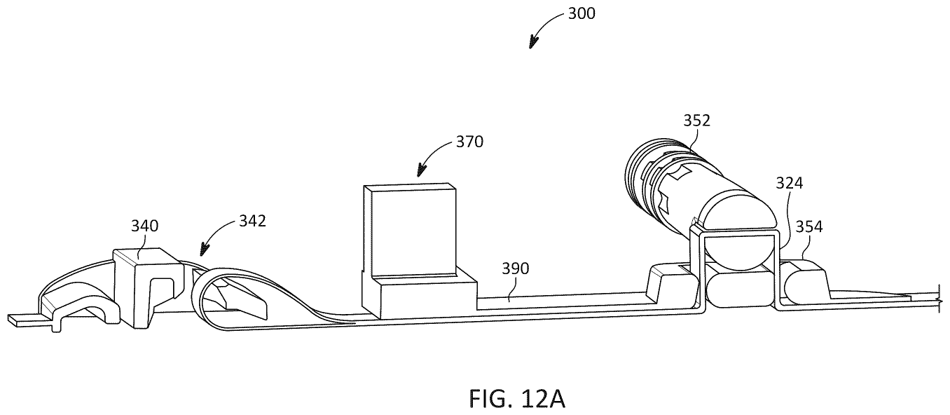

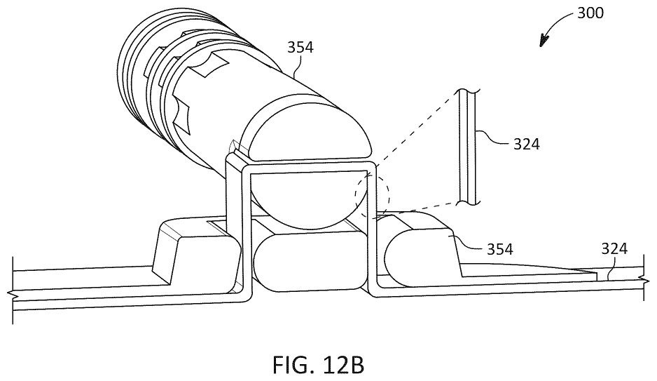

In the embodiment FIG. 12A and FIG. 12B, a tourniquet 300 comprises a tourniquet band and hardware, such as a tightening mechanism (e.g., windlass 352), a strap guide 354, a securing mechanism 370, and buckle 340. In the illustrated embodiment, strap guide 354 and securing mechanism 370 may be integrated into a base plate 290 (e.g., a windlass base), which may be similar to base plate 90 with a strap guide and one or more windlass hooks with clip—in some embodiments—but without buckle 40. Buckle 340 is provided as a separate piece of hardware from the windlass base.

The tourniquet band may comprise an outer sleeve (not shown) and an inner strap 324. The outer sleeve may be constructed as discussed above with respect to outer sleeve 22. The inner band 324 includes a first end and a second end (not shown). A portion of inner strap 324 is anchored to the outer sleeve as for example, by sewing, glueing, stapling, clamping, or heat/ultra-sound (sonic) welding, or another anchoring mechanism. According to an even more particular embodiment, the second end of inner strap 324 is anchored to the outer sleeve at, for example, the second end of outer sleeve (i.e., at the tail end of outer sleeve).

In one embodiment, inner strap 324 exits the outer sleeve of tourniquet band, passes through the strap guide 354, which may be similar to strap guide 54, through the windlass 352, through the next slot in the strap guide 354 and into an area between the lower cover portion of outer sleeve and base member 292. The lower portion of outer sleeve and inner strap 224 loop through and attachment slot 242 of buckle 240. The end of inner strap 224 that looped through buckle 240 may be routed back through strap guide 354, windlass 352, the other slot of strap guide 354 and back into the interior pocket of the outer sleeve. This end of the inner strap 324 may be anchored to inner strap 324 at a location along the length of inner strap 324 between the windlass base (e.g., base plate 390) and the free end of the windlass strap, as for example by sewing, glueing, stapling, clamping, or heat/ultra-sound (sonic) welding, or another anchoring mechanism. The portion of the outer sleeve that looped through attachment slot 242 may double back and be attached back to the outer sleeve between buckle 240 and the windlass base.

FIGS. 12A and 12B are similar to the embodiment of FIG. 11, but with the inner strap doubled back on itself to double route inner strap 324 through the slots of strap guide 354 and windlass 352. In some embodiments, the end of inner strap 324 that was routed through the buckle 340 is anchored to inner strap 324 part way between the windlass base and the other end of inner strap 324. In other embodiments, the end of inner strap 324 that was routed through buckle 340 and back is anchored back to the other end of the inner strap or at or near the tip of the outer sleeve/tourniquet band to form a continuous loop.

It will be appreciated that one or more of the elements depicted in the drawings/figures can also be implemented in a more separated or integrated manner, or even removed or rendered as inoperable in certain cases, as is useful in accordance with a particular application. Additionally, any signal arrows in the drawings/figures should be considered only as exemplary, and not limiting, unless otherwise specifically noted.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited to only those elements but may include other elements not expressly listed or inherent to such process, product, article, or apparatus.

Furthermore, the term “or” as used herein is generally intended to mean “and/or” unless otherwise indicated. For example, a condition A or B is satisfied by any one of the following: A is true (or present) and B is false (or not present), A is false (or not present) and B is true (or present), and both A and B are true (or present). As used herein, a term preceded by “a” or “an” (and “the” when antecedent basis is “a” or “an”) includes both singular and plural of such term, unless clearly indicated within the claim otherwise (i.e., that the reference “a” or “an” clearly indicates only the singular or only the plural). Also, as used in the description herein and throughout the meaning of “in”includes “in”and “on”unless the context clearly dictates otherwise.

Reference throughout this specification to “one embodiment”, “an embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

Additionally, any examples or illustrations given herein are not to be regarded in any way as restrictions on, limits to, or express definitions of, any term or terms with which they are utilized. Instead, these examples or illustrations are to be regarded as being described with respect to one particular embodiment and as illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” “in one embodiment.”

Thus, while the invention has been described with respect to specific embodiments thereof, these embodiments are merely illustrative, and not restrictive of the invention. Rather, the description is intended to describe illustrative embodiments, features and functions in order to provide a person of ordinary skill in the art context to understand the invention without limiting the invention to any particularly described embodiment, feature or function, including any such embodiment feature or function described. While specific embodiments of, and examples for, the invention are described herein for illustrative purposes only, various equivalent modifications are possible within the spirit and scope of the invention, as those skilled in the relevant art will recognize and appreciate.

As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component.

What is claimed IS:

1. A tourniquet for restricting a flow of blood in a body part, the tourniquet comprising:
a tourniquet band, the tourniquet band having a tail end, the tourniquet band comprising:
an outer sleeve;
an inner tightening strap in slidable engagement with the outer sleeve, wherein a portion of the inner tightening strap is anchored to the outer sleeve;
a windlass operable to tighten the inner tightening strap;
a windlass base, the windlass base comprising a strap guide defining a first guide slot and a second guide slot to route the inner tightening strap to and from the windlass, wherein the inner tightening strap is routed from the outer sleeve and through the windlass base to the windlass through the first guide slot, through the windlass, from the windlass to the second guide slot, and through the windlass base via the second guide slot, wherein the strap guide is configured to allow smooth movement of the inner tightening strap during operation of the windlass to allow the windlass to steadily build occlusion pressure beginning with a first rotation of the windlass; and
a buckle defining an adjustment slot adapted for receiving the tail end of the tourniquet band.

2. The tourniquet of claim 1, wherein the inner tightening strap is single routed through the first guide slot, the windlass, and the second guide slot.

3. The tourniquet of claim 2, wherein the inner tightening strap is routed from the second guide slot back into the outer sleeve to double back on itself.

4. The tourniquet of claim 3, wherein the inner tightening strap comprises a first end and a second end, wherein the second end of the inner tightening strap is anchored to the outer sleeve and wherein the first end of the inner tightening strap is anchored back to the inner tightening strap.

5. The tourniquet of claim 4, wherein the first end of the inner tightening strap is anchored back to the inner tightening strap away from the second end of the inner tightening strap.

6. The tourniquet of claim 5, wherein the outer sleeve comprises a first end and a second end, wherein the second end of the inner tightening strap is anchored to the second end of the outer sleeve and wherein the windlass base is coupled to the first end of the outer sleeve.

7. The tourniquet of claim 1, wherein the windlass base is overmolded to the outer sleeve.

8. The tourniquet of claim 1, wherein the first guide slot and the second guide slot are longitudinally spaced to be wider than a diameter of the windlass where the inner tightening strap passes through the windlass.

9. The tourniquet of claim 1, wherein the strap guide defines radiused entrance and exit features to the first guide slot and the second guide slot to accommodate changes in an exit angle of the inner tightening strap due to longitudinal movement of the windlass.

10. The tourniquet of claim 9, wherein at least one of the radiused entrance or exit features extends an entire depth the first guide slot or the second guide slot.

11. The tourniquet of claim 1, wherein the strap guide comprises:
a first longitudinal portion;
a second longitudinal portion;
a first lateral portion spanning between the first longitudinal portion and the second longitudinal portion;
a second lateral portion spanning between the first longitudinal portion and the second longitudinal portion; and
an intermediate lateral portion spanning between the first longitudinal portion and the second longitudinal portion, the intermediate lateral portion located between the first lateral portion and the second lateral portion, wherein the first longitudinal portion, the second longitudinal portion, the first lateral portion and the intermediate lateral portion define the first guide slot, and wherein the first longitudinal portion, the second longitudinal portion, the second lateral portion and the intermediate lateral portion define the second guide slot.

12. The tourniquet of claim 11, wherein the first lateral portion defines a lateral guide wall facing the first guide slot, wherein the lateral guide wall comprises a straight segment that is tilted relative a z-axis that is perpendicular to a longitudinal axis and a lateral axis of the tourniquet.

13. The tourniquet of claim 12, wherein the lateral guide wall has a radiused inner end and a radiused outer end.

14. The tourniquet of claim 1, wherein rotating the windlass takes up length of the inner tightening strap in one longitudinal direction relative to the windlass.

15. The tourniquet of claim 1, further comprising a securing mechanism.

16. The tourniquet of claim 15, wherein at least of the buckle or the securing mechanism is integrated with the windlass base.

17. The tourniquet of claim 15, wherein the securing mechanism comprises:
a pair of hooked catches configured to retain the windlass in a tensioned position;
a clip coupled to a first hooked catch from the pair of hooked catches and configured to engage a second hooked catch from the pair of hooked catches to further secure the windlass.

18. The tourniquet of claim 17, wherein the clip comprises a metal wire form and is rotatable between an open position and a closed position.

19. The tourniquet of claim 18, wherein the second hooked catch includes a retaining feature to lock the clip in the closed position.

20. The tourniquet of claim 1, wherein the buckle is overmolded to the tourniquet band.
